(19) United States
(12) Patent Application Publication
(10) Pub. No.: US 2003/0228253 A1
(43) Pub. Date: Dec. 11, 2003

(54) Radiolabelled thioplatin, compositions thereof and methods of cancer treatment

(76) Inventors: Shraga D. Aranoff, Flushing, NY (US); Jack Schwartzberg, Old Westbury, NY (US); Stanley E. Order, Owings Mills, MD (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/184,543

(22) Filed: Jun. 28, 2002

Related U.S. Application Data
(60) Provisional application No. 60/386,592, filed on Jun. 6, 2002.

(51) Int. Cl. A61K 51/00

(52) U.S. Cl. 424/1.11

(57) ABSTRACT

The present invention relates to radioactive platinum compounds, pharmaceutical compositions containing radioactive platinum compounds and methods of treating cancer using the compounds and compositions.

### RELATED APPLICATIONS

This application claims the benefit of the filing date of provisional application filed on Jun. 6, 2002, the entire disclosure of which is incorporated by reference herein.

### BACKGROUND OF THE INVENTION

This invention relates to radioactive platinum compounds, pharmaceutical compositions containing radioactive platinum compounds and methods of treating cancer using the compounds and compositions.

In Areberg, J., et al., Antitumor Effect of Radioactive ¹⁹¹Cisplatin on Nude Mice, Vol. 49, #3, p. 827-832 (2001), Areberg reported in the International Journal of Radiation Oncology, Biology and Physics that nude mice bearing a human squamous carcinoma could be inhibited by ¹⁹¹cisplatin which was cytotoxic to the tumor by evaluating tumor growth delay, log of tumor growth and area under the curve of the log of tumor growth. He further suggested that ^{193m}cisplatin or ^{195m}cisplatin could be used as isotopes of cisplatin, although he did not utilize these in his experiments.

Thioplatin distinguishes itself from cisplatin in that it contains sulfur groups which inhibit the peripheral toxicity as seen with cisplatin. This was demonstrated by direct biopsy of the kidney and intestinal tract, and in contrast where toxicity had been noted with cisplatin, it was not with thioplatin. Thus, an intrinsic feature of thioplatin is the lack of normal tissue toxicity when compared to cisplatin. In addition, the binding of DNA of cisplatin is limited to 14.3 nanograms/milligrams, whereas thioplatin binds almost one-thousand fold greater.

In U.S. Pat. No. 6,074,626, the entire disclosure of which is incorporated by reference herein, is disclosed methods of treating cancer using radioactive cisplatin.

### DETAIL DESCRIPTION OF THE PREFERRED EMBODIMENTS

Thioplatin is a sulfur-containing complex which is most effective at an acidic pH. Glucose metabolism of solid tumors converts glucose anaerobically to lactic acid; therefore causing a pH of 6.8 a drop from the normal pH of 7.2 to 7.4. Thioplatin then acts at an acidic pH optimum and is cytotoxic for tumor cells. Using human tumor xenografts in nude mice, significant tumor cytotoxicity has been demonstrated with reduced normal tissue toxicity when compared to the standard of cisplatin, itself.

Thioplatin administered to experimental animals was assayed for the effects of the drug on body weight, white blood cells, BUN and histophathology of kidneys and small intestines. All were negligible while being cytotoxic to human lung and colon cancer. The predicated increased cytotoxicity was demonstrated to be 5 to 10 times greater than cisplatin. Binding by thioplatin is 30 to 100 times greater at the tumor DNA when compared to cisplatin.

Platinum may be made radioactive by a variety of techniques and the present disclosure is not limited merely to those platinum complexes identified herein which can complex with thioplatin. Neutron bombardment of ¹⁹⁴platinum yields ^{195m}platinum which can be incorporated into thioplatin chemically. In addition, other isotopes of platinum such as ^{193m}platinum and ¹⁹¹platinum can also be linked to thioplatin.

^{195m}Platinum is cytotoxic by its release of “auger” electron at 33/disintegrations and with a half-life of over 4 days. In order to amplify the cytotoxicity of the tumor cells, the final drug radioactive thioplatin must be located on the DNA of tumor cells. For cisplatin at a pH of 6.8 (solid tumor pH norm), DNA binding occurred at 14.6 ng/mg, whereas thioplatin at a pH 6.8 DNA binding was 13,870 ng/mg. Radioactive thioplatin can be quantitated in the clinic by nuclear SPECT scanning of the tumor and normal tissues for low-energy photons (65-129 kev) emitted by radioactive platinum.

Based on pre-clinical experience, the cytotoxicity of thioplatin is 5 to 10 times greater than the standard drug for cancer chemotherapy, i.e., cisplatin. The predicted amplification of cytotoxicity by making thioplatin radioactive is 4.8 times the relative biologic effect of 250 kev x-rays. Thus, from 25 to 50 times the cytotoxicity of standard cisplatin is expected from radioactive thioplatin. The administration of radioactive thioplatin may be intra-arterial by direct tumor infusion, intraperitoneally or intravenously administered by methods and use of compositions known to those skilled in the art of radiopharmaceuticals. Thus, the added feature of a reduced normal tissue toxicity and amplification of tumor cell toxicity at an acidic pH has been demonstrated in pre-clinical testing and makes the use of radioactive thioplatin highly attractive and more powerful for cancer cytotoxicity than the drug itself.

A typical dose for the compounds of the present invention depends on the route of administration and the patient size. A typical dose could be approximately 10 millicuries or more. Further refinement of the dosage could be elucidated in Phase I clinical trials using the formulations of the present invention.

Administration of radioactive cisplatin may be accomplished as described in U.S. Pat. No. 6,074,626, the entire disclosure of which is incorporated by reference herein. Methods of administration of radioactive platinum complexes with thioplatin may be similar to those described in this patent or modified depending upon the conditions of use by one skilled in the art.

Compositions in accordance with the present invention may be administered with carriers known to those skilled in the art for typical cancer therapies and may include saline solutions, buffer solutions for injectable and oral solutions and solid carriers such as binders, sugars, starches, celluloses, etc. where appropriate, depending upon the circumstances of use.

In addition to making thioplatin radioactive by complexing with one or a combination of radioactive platinum(s), other chemo therapeutic agents as well may be made more effective by complexing with radioactive platinum and are thus considered part of the present invention.

Without further elaboration the foregoing will so fully illustrate our invention that others may, by applying current or future knowledge, adapt the same for use under various conditions of service.

We claim:

1. A method of treating cancer by administering to a living being a therapeutically effective amount of a composition comprising radiolabelled thioplatin.

2. The method of claim 2 additionally comprising the step of selecting the radiolabelled thioplatin to comprise a radioactive platinum compound.

3. The method of claim 2 wherein the radioactive platinum is ^{195m}platinum ^{193m}platinum and/or ¹⁹¹platinum.

4. The method of claim 1 additionally comprising the step of selecting the composition to comprise a therapeutically acceptable carrier material.

5. A pharmaceutical composition for treating cancer comprising radiolabelled thioplatin.

6. The composition of claim 5 additionally comprising a carrier material.

7. The composition of claim 5 wherein the radiolabelled thioplatin is radiolabelled with radioactive platinum.

8. The composition of claim 7 wherein the platinum is ^{195m}platinum ^{193m}platinum and/or ¹⁹¹platinum.
